(19) United States
(12) Patent Application Publication
(10) Pub. No.: US 2012/0252894 A1
(43) Pub. Date: Oct. 4, 2012

(54) COMPOUNDS FOR ALLEVIATING PAIN AND STRESS IN FETUS AND NEWBORN

(76) Inventors: Giniatullin Rashid, Marseille (FR); Khazipov Roustem, Marseille (FR); Ben-Ari Yehezkel, Marseille (FR); Mazzuca Michel, Marseille (FR); Tyzio Roman, Marseille (FR)

(21) Appl. No.: 13/435,522

(22) Filed: Mar. 30, 2012

Related U.S. Application Data
(60) Provisional application No. 61/470,631, filed on Apr. 1, 2011.

(51) Int. Cl. A61K 31/195; G01N 33/567; A61P 29/00; C07C 311/44

(52) U.S. Cl. 514/562; 562/430; 435/7.21

(57) ABSTRACT

The invention relates to a compound which inhibits the importation of chloride into neurons or a compound which improve the outflow of chloride from neurons thereby promoting the inhibitory actions of GABA and alleviating pain and stress of the fetus during delivery and the newborn. The invention also relates to a pharmaceutical composition for use in a method for alleviating pain and stress of the fetus during delivery and the newborn comprising a compound according to the invention and a pharmaceutically acceptable carrier.

### CROSS-REFERENCE TO RELATED APPLICATIONS

This application claims priority to U.S. Provisional Patent Application Ser. No. 61/470,631 filed Apr. 1, 2011. The substance of that application is herein incorporated by reference.

### FIELD OF THE INVENTION

The invention relates to a compound which inhibits the importation of chloride into neurons or a compound which improve the outflow of chloride from neurons thereby promoting the inhibitory actions of GABA and alleviating pain and stress of the fetus during delivery and the newborn.

### BACKGROUND OF THE INVENTION

Delivery is stressful and potentially painful event for the newborn. Sources of pain during delivery can be natural, such as severe mechanical compression of the fetus during his passage via delivery channel (Derek, 1999), and iatrogenic, such as forceps extraction, blood samples and injections. Clinical studies indicate that painful experiences in neonates may disrupt the adaptation of newborn infants to their postnatal environment and in the long term, lead to psychological sequelae. In mice, early exposure to noxious or stressful stimuli alters pain sensitivity and behaviour in adult life, possibly by altering the stress-axis and antinociceptive circuitry. Therefore, the problem of pain in the newborn is of clinical importance; however, the mechanisms involved in pain regulation at birth are poorly understood.

Recently, comparison of the pain responses in human neonates born with vaginal delivery and or planned caesarean section revealed diminished physiological, behavioural and vocalization responses to the painful stimuli following vaginal delivery when compared to C sections, suggesting that antinociceptive analgesic mechanisms are activated and last for few hours during and after normal delivery. The mechanisms underlying this transient newborn analgesia at present remain unknown. The inventors have recently discovered that the hormone oxytocin that triggers delivery and exerts multiple actions in the nervous system also has an analgesic action. In adult rats, oxytocin exerts analgesic action. Analgesic effect of oxytocin in adults is mediated by GABAergic inhibition of the nociceptive inputs to the dorsal horn of the spinal cord. On the other hand, nociception is strongly regulated not only by amount of the GABA(A) receptor mediated anionic conductance, but also by its reversal potential (EGABA), and depolarizing shifts in EGABA in the nociceptive and dorsal horn neurons are associated with elevated pain (De Koninck, 2007; Price et al., 2008). Pain is alleviated by pharmacological blockade or genetic knock out of NKCC1 chloride co-transporter, which is the primary cause for elevated chloride and depolarizing action of GABA in the nociceptive neurons. In an attempt to determine how oxytocin acts, the inventors discovered that in immature cortical neurons, oxytocin and NKCC1 blockers like bumetanide produce similar negative shift in EGABA (Tyzio et al., 2006; Khazipov et al., 2008) suggesting common mechanisms of action. The inventors then showed that the hormone like the NKCC1 diuretic antagonist bumetanide exert an analgesic action by reducing intracellular chloride in pain pathways thereby enhancing the inhibitory actions of GABA and reducing pain.

### SUMMARY OF THE INVENTION

Oxytocin and diuretics are already known to have an analgesic action in adults although neither their mechanisms of action nor the possible links between them was established. The inventors described for the first time the analgesic action at that age, during this process and the intervention of NKCC1 in both of these events. In essence, the study unravels a mechanism of action by which during a precise limited period, oxytocin released to trigger labour also endogenously acts to protect the newborn from excessive pain via inactivation of NKCC1 co-transporter.

Thus the invention relates to a compound for use in a method for alleviating pain and stress in fetus and newborn.

In another aspect, the invention relates to a pharmaceutical composition for use in a method for alleviating pain and stress in fetus and newborn comprising a compound according to the invention and a pharmaceutically acceptable carrier.

### DETAILED DESCRIPTION OF THE INVENTION

### Definitions

Throughout the specification, several terms are employed and are defined in the following paragraphs.

As used herein, the term “newborn” denotes a period beginning after birth and lasting through the 28^{th} day following birth.

As used herein, the term “preterm baby” denotes a baby of less than 37 weeks gestational age.

As used herein, “NKCC” for “Na-K-C1 co-transporter” denotes a protein that assists in the active transport of sodium, potassium, and chloride into and out of cells. There are several varieties, or isoforms, of this membrane transport protein, notably NKCC1 and NKCC2. NKCC1 is widely distributed throughout the body but also in the brain and in particular in the developing animal and human brain. It acts to augment intracellular chloride in neurons and thereby to render GABA more excitatory. Extensive investigations indicate that blocking NKCC1 reduce intracellular chloride thereby augmenting the inhibitory actions of GABA. In vivo and in vitro studies have now indicated that genetic and/or pharmacological blockade of NKCC1 reduces early network activity.

As used herein, the term “KCC” for “potassium chloride co-transporter” denotes a co-transporter of chloride. There are several varieties, or isoforms, notably KCC2. KCC2 is found in many organs notably in the brain acts to remove intracellular chloride and thereby to augment the inhibitory actions of GABA. Blockers of KCC2 transform GABA to excitatory and facilitate the generation of seizures and genetic invalidation of KCC2 is lethal in mice. KCC2 is also expressed relatively late in development paralleling the shift of the actions of GABA from excitatory to inhibitory. Also, a wide range of insults and seizures remove functional KCC2 thereby leading to persistent excitatory actions of GABA and further seizures.

As used herein, the term “diuretic” denotes any drug that elevates the rate of urination and thus provides a means of forced diuresis. There are several categories of diuretics. All diuretics increase the excretion of water from bodies, although each class does so in a distinct way.

As used herein, the term “loop diuretics” denotes diuretics that act on the ascending loop of Henle in the kidney.

As used herein, the term “alleviating” denotes reversing, inhibiting the progress of, or preventing the disorder or condition to which such term applies, or reversing, inhibiting the progress of, or preventing one or more symptoms of the disorder or condition to which such term applies.

### Antagonists and Uses Thereof

A first object of the invention relates to a compound which inhibits the importation of chloride into neurons or a compound which improve the outflow of chloride from neurons for use in a method for alleviating pain and stress in fetus and newborn.

In a preferred embodiment, the compound according to the invention inhibits the NKCC co-transporter or activates the KCC co-transporter.

In another preferred embodiment, the compound according to the invention is an antagonist of NKCC co-transporter or an agonist of KCC co-transporter.

In another embodiment, the compound or the pharmaceutical composition according to the invention is administered to the fetus during delivery or to the newborn in his first hours of life.

In another preferred embodiment, the compound or the pharmaceutical composition according to the invention is administered to the newborn in his 2 first hours of life.

In another preferred embodiment, the compound or the pharmaceutical composition according to the invention is administered to the newborn in his 10 first hours of life.

In another preferred embodiment, the compound or the pharmaceutical composition according to the invention is administered to the newborn in his 24 first hours of life.

In another preferred embodiment, the compound or the pharmaceutical composition according to the invention is administered to a preterm baby.

In another embodiment, the compound or the pharmaceutical composition according to the invention is administered to the mother during delivery.

In another embodiment, the compound or the pharmaceutical composition according to the invention is administrated to the mother during a caesarean section delivery.

In one embodiment, said NKCC antagonist or KCC agonist may be a low molecular weight antagonist, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules have a size range up to about 5000 Da, more preferably up to 2000 Da, and most preferably up to about 1000 Da.

In preferred embodiment, the compound which inhibits the NKCC co-transporter is a diuretic.

In another preferred embodiment, the diuretic is a loop diuretic.

In a preferred embodiment, the compound according to the invention is selected from the group consisting of: bumetanide, furosemide, ethacrynic acid, torsemide, azosemide, muzolimine, piretanide, tripamide and the like; thiazide and thiazide-like diuretics, such as bendroflumethiazide, benzthiazide, chlorothiazide, hydrochlorothiazide, hydro-flumethiazide, methylclothiazide, polythiazide, trichlormethiazide, chlorthalidone, indapamide, metolazone and quinethazone; and analogs and functional derivatives of such compounds.

In another preferred embodiment, the compound according to the invention is bumetanide.

In a preferred embodiment, an analog of the bumetanide according to the invention may have a formula as described in the patent application WO2010085352.

In a preferred embodiment, the analog of the bumetanide may be bumetanide aldehyde, bumetanide dibenzylamide, bumetanide diethylamide, bumetanide morpholinoethyl ester, bumetanide 3-(dimethylaminopropyl) ester, bumetanide N,N-diethylglycolamide ester, bumetanide dimethylglycolamide ester, bumetanide pivaxetil ester, bumetanide methoxy(polyethyleneoxy)n-i-ethyl ester,_bumetanide benzyltrimethyl-ammonium salt, and bumetanide cetyltrimethylammonium salt.

In another preferred embodiment, the analog of the bumetanide may be furosemide aldehyde, furosemide ethyl ester, furosemide cyanomethyl ester, furosemide benzyl ester, furosemide morpholinoethyl ester, furosemide 3-(dimethylaminopropyl) ester, furosemide N,N-diethylglycolamide ester, furosemide dibenzylamide, furosemide benzyltrimethylammonium salt, furosemide cetyltrimethylammonium salt, furosemide N,N-dimethylglycolamide ester, furosemide methoxy(polyethyleneoxy)n-i-ethyl ester, furosemide pivaxetil ester and furosemide propaxetil ester.

In another preferred embodiment, the analog of the bumetanide may be piretanide aldehyde, piretanide methyl ester, piretanide cyanomethyl ester, piretanide benzyl ester, piretanide morpholinoethyl ester, piretanide 3-(dimethylaminopropyl) ester, piretanide N,N-diethylglycolamide ester, piretanide diethylamide, piretanide dibenzylamide, piretanide benzylltrimethylammonium salt, piretanide cetylltrimethylammonium salt, piretanide N,N-dimethylglycolamide ester, piretanide methoxy(polyethyleneoxy)n-i-ethyl ester, piretanide pivaxetil ester and/or piretanide propaxetil ester.

In another preferred embodiment, the analog of the bumetanide may be tetrazolyl-substituted azosemides (such as methoxymethyl tetrazolyl-substituted azosemides, methylthiomethyl tetrazolyl-substituted azosemides and N-mPEG350-tetrazolyl-substituted azosemides), azosemide benzyltrimethylammonium salt and/or azosemide cetyltrimethylammonium salt.

In another preferred embodiment, the analog of the bumetanide may be pyridine-substituted torsemide quaternary ammonium salts or the corresponding inner salts (zwitterions). Examples include, but are not limited to, methoxymethyl pyridinium torsemide salts, methylthiomethyl pyridinium torsemide salts and N-mPEG350-pyridinium torsemide salts.

In another embodiment, the compound according to the invention is the oxytocin.

In another embodiment, NKCC antagonist or KCC agonist of the invention may consist in an antibody which inhibits NKCC or activates KCC or an antibody fragment which inhibits NKCC or activates KCC.

Antibodies directed against NKCC or KCC can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against NKCC or KCC can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see, e.g., U.S. Pat. No. 4,946,778) can be adapted to produce anti-NKCC or anti-KCC single chain antibodies. NKCC antagonists or KCC agonists useful in practicing the present invention also include anti-NKCC antibody fragments or anti-KCC antibody fragment including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity to NKCC or KCC.

Humanized anti-NKCC antibodies or anti-KCC antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In still another embodiment, NKCC antagonists or KCC agonists may be selected from aptamers. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

In another preferred embodiment, the compound according to the invention is an inhibitor of the NKCC co-transporter expression.

Small inhibitory RNAs (siRNAs) can also function as inhibitors of NKCC co-transporter gene expression for use in the present invention. NKCC co-transporter gene expression can be reduced by contacting a subject or cell with a small double stranded RNA (dsRNA), or a vector or construct causing the production of a small double stranded RNA, such that NKCC co-transporter gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of NKCC co-transporter gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of NKCC co-transporter mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of NKCC co-transporter gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing NKCC co-transporter. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In a preferred embodiment, the compound according to the invention is a KCC2 agonist.

In a preferred embodiment, the compound according to the invention is a compound which inhibits the level of the NKCC protein on the neuron surface or improves the level of the KCC protein on the cell surface.

Another object of the invention relates to a method for alleviating pain and stress in fetus and newborn comprising administering to a subject in need thereof with a compound which inhibits the importation of chloride into neurons or a compound which improve the outflow of chloride from neurons.

In one aspect, the invention relates to a method for alleviating pain and stress in fetus and newborn comprising administering to a subject in need thereof a NKCC antagonist as above described.

In another aspect, the invention relates to a method for alleviating pain and stress in fetus and newborn comprising administering a compound selected from the group consisting of: bumetanide, furosemide, ethacrynic acid, torsemide, azosemide, muzolimine, piretanide, tripamide and the like; thiazide and thiazide-like diuretics, such as bendroflumethiazide, benzthiazide, chlorothiazide, hydrochlorothiazide, hydro-flumethiazide, methylclothiazide, polythiazide, trichlormethiazide, chlorthalidone, indapamide, metolazone and quinethazone; and analogs and functional derivatives of such compounds.

In another aspect, the compound is bumetanide.

Compounds of the invention may be administered in the form of a pharmaceutical composition, as defined below.

Preferably, said compound which inhibits the importation of chloride into neurons or which improve the outflow of chloride from neurons, preferably said antagonist of NKCC or said agonist of KCC, is administered in a therapeutically effective amount.

By a “therapeutically effective amount” is meant a sufficient amount of compound to treat and/or to prevent diseases as described previously.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Compounds according to the invention may be used for the preparation of a pharmaceutical composition for use in a method for alleviating pain and stress in fetus and newborn.

Hence, the present invention also provides a pharmaceutical composition comprising an effective dose of a compound which inhibits the NKCC co-transporter, preferably a NKCC antagonist or which activates the KCC co-transporter, according to the invention.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

In a preferred embodiment, the pharmaceutical composition may be administrated by intranasal spray.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical composition according to the invention may also contain other compounds, which may be biologically active or inactive. For example, one or more treatment agents of the present invention may be combined with another agent, in a treatment combination, and administered according to a treatment regimen of the present invention. Such combinations may be administered as separate compositions, combined for delivery in a complementary delivery system, or formulated in a combined composition, such as a mixture or a fusion compound. Additionally, the aforementioned treatment combination may include a BBB permeability enhancer and/or a hyperosmotic agent.

Alternatively, compounds of the invention which inhibits the NKCC co-transporter or activates the KCC co-transporter can be further identified by screening methods as hereinafter described.

### Screening Methods

Another object of the invention relates to a method for screening a compound which inhibits the NKCC co-transporter of activates the KCC co-transporter.

In particular, the invention provides a method for screening a NKCC antagonist or a KCC agonist for the treatment of pain and stress the fetus during delivery and the newborn.

For example, the screening method may measure the binding of a candidate compound to NKCC or KCC, or to cells or membranes bearing NKCC or KCC or a fusion protein thereof by means of a label directly or indirectly associated with the candidate compound. Alternatively, a screening method may involve measuring or, qualitatively or quantitatively, detecting the competition of binding of a candidate compound to the receptor with a labelled competitor (e.g., antagonist).

Furthermore, screening methods may test whether the candidate compound results in a signal generated by an antagonist of NKCC or an agonist of KCC, using detection systems appropriate to cells bearing the receptor.

In a particular embodiment, the screening method of the invention comprises the step consisting of:

a) providing neurons expressing NKCC or KCC on their surface:

b) incubating said cells with a candidate compound;

c) determining whether said candidate compound binds to and inhibits NKCC or binds to and activates KCC; and

d) selecting the candidate compound that binds to and inhibits NKCC or binds to and activates KCC.

In one embodiment, the NKCC co-transporter or the KCC co-transporter used in the screening method may be its orthologs and derivatives as defined in the present invention.

In general, such screening methods involve providing appropriate cells which express NKCC or KCC, its orthologs and derivatives thereof on their surface. In particular, a nucleic acid encoding NKCC or KCC may be employed to transfect cells to thereby express the receptor of the invention. Such a transfection may be accomplished by methods well known in the art.

In a particular embodiment, cells are selected from the group consisting of glial cells, neuronal cells, neurones, transfected cell lines for investigations or renal cells of any species (mouse, human . . . ).

The screening method of the invention may be employed for determining an antagonist or agonist by contacting such cells with compounds to be screened and determining whether such compound inhibits or activates the co-transporter.

The determination of the inhibition of NKCC can be assessed by determining the cell viability. A compound is deemed to decrease cell viability if it is negative in any one the methods described below as examples of cell rescue activity.

According to a one embodiment of the invention, the candidate compound of may be selected from a library of compounds previously synthesised, or a library of compounds for which the structure is determined in a database, or from a library of compounds that have been synthesised de novo or natural compounds.

The candidate compound may be selected from the group of (a) proteins or peptides, (b) nucleic acids and (c) organic or chemical compounds (natural or not). Illustratively, libraries of pre-selected candidate nucleic acids may be obtained by performing the SELEX method as described in documents U.S. Pat. No. 5,475,096 and U.S. Pat. No. 5,270,163. Further illustratively, the candidate compound may be selected from the group of antibodies directed against NKCC or KCC.

Such the method may be used to screen NKCC antagonists or KCC agonists according to the invention.

The invention will be further illustrated by the following examples. However, these examples should not be interpreted in any way as limiting the scope of the present invention.

### Example

### Material & Methods

Animals

All animal use protocols conformed to the INSERM guidelines and the Italian act Decreto Legislativo 27 Jan. 1992 n. 116 implementing the European Community directives n. 86/609 and 93/88 on the use of laboratory animals. Pregnant and maternal Wistar rats were housed with a 12-h light-dark-cycle, at 24°±1° C., and food and water ad libitum. The day of birth was considered 0 day old (P0). Experiments on P0 rats (male and female) were performed within two hours after birth.

Quantification of the Nociceptive Response

Tail Immersion

The pup was held in a box with a hole allowing the tail to protrude from it. The inner surface of the box was covered with an aluminum sheet forming an electrical contact with the rat body. The electrical circuit via the sheet, body, tail and water bath was powered by a 1.5 V battery and its connection and disconnection could be easily detected upon the tail immersion and withdrawal from the water, respectively. Electrical signals were digitized at 1 kHz using a Digidata 1200 and recorded to a computer. The distal tip of the tail was lowered into the water bath (50° C.). Latency to withdrawal was recorded as the “pain” parameter, with a 15-second maximum allowable threshold. After three habituation tests, the latency to withdrawal was determined from the average of three consecutive measurements.

Vocalization

Under isoflurane (1.5%) anaesthesia, rat pups were implanted with bipolar electrodes into the whisker pad and decerebrated at the upper pons level via a hole drilled 1 mm posterior to lambda using blunted 36 gouge needle, to avoid blood bleeding the hole was covered by cyanoacrylate after. After 10 min (P0) or one hour (P1-2 pups) of recovery period, the pups were wrapped by cotton and placed on the thermal blanket (38° C.). Whisker pad was stimulated by electrical train pulses (1 ms pulse duration, 5-25 V amplitude, 50 Hz, 1 minute inter-train interval). Vocalization response was recorded by microphone, digitized at 10 kHz using Digidata 1440 interface (Axon Instruments) and analyzed offline using Matlab (MathWorks, Natick, Mass.). To quantify the vocalization response, we calculated scalar integral (□) as following: (i) raw acustogram was converted to scalar acustogram by inverting all negative values to positive values; (ii) scalar acustogram was corrected for the baseline activity level by subtraction of the mean scalar acustogram value calculated during 1 minute before stimulation; (iii) scalar acustogram integral was calculated as cumulative, corrected for the baseline, scalar acustogram during 5 sec after stimulation.

Calcium Imaging

Trigeminal sensory neurons were obtained from P0 rats. Animals were anesthetized by CO2 and decapitated. Trigeminal ganglia were excised and enzymatically dissociated in F12 medium containing 0.25 mg/ml trypsin, 1 mg/ml collagenase and 0.2 mg/ml DNAse (Sigma) at 37° C. Cells were plated on poly-1-lysine-coated Petri dishes in F12 medium with 10% fetal calf serum and examined 5 hours after plating. For Ca2+ imaging experiments cells were incubated for 40 min at 20-22° C. in physiological solution containing Fluo3 (AM ester cell-permeable compound; 1 μM; Molecular Probes), followed by a 30 min washout period. Fluorescence emission was detected with a Cell-R imaging system (Olympus, Hamburg, Germany). Images were acquired with 200 ms exposure time and single cell responses were analyzed with the Cell-R software. All drugs were applied via fast perfusion system (RDS-200, BioLogic Science Instruments Grenoble, France). Only cells with two stable control GABA transients were taken into analysis. Intracellular Ca2+ transients were expressed as percent amplitude increase (ΔF/F0, where F0 is the baseline fluorescence level and ΔF is the increment over baseline). Ca2+ transient intensity data was exported and then analyzed off-line using Excel and Origin (version 8.0) software. Significance was analyzed by non-parametric Mann-Witney test.

Single GABA Channel Recordings

Single GABA channel recordings were performed from the trigeminal sensory neurons prepared as described above. Cell attached patch-clamp recordings were performed using Axopatch 200A (Axon Instruments, Union City, Calif.) and EPC-9 (HEKA Elektronik Dr. Schulze GmbH, Lambrecht/Pfalz, Germany) amplifiers. Patch electrodes were made from borosilicate glass capillaries (GC150F-15, Clark Electromedical Instruments). For recordings of single GABA(A) channels, patch pipette solution contained (in mM): NaCl 120, TEA-Cl 20, KCl 5, 4-aminopyridine 5, CaCl2 0.1, MgCl2 10, glucose 10, Hepes-NaOH 10 buffered to pH 7.2-7.3 and GABA (1-5 μM) was added at the day of experiment from 1 mM frozen stock solution. Driving force for GABA(A) receptor mediated currents was determined from the current-voltage relationships of the currents through single GABA(A) channels single as described earlier (Tyzio et al., 2006) and corrected for an error of 2 mV (Tyzio et al., 2008).

Primary Afferents Depolarization

Experiments were performed on lumbar (L) spinal cord preparations isolated from neonatal Wistar rats (P0-P1). All efforts were made to reduce the number of animals used and to minimize animal suffering. The experimental setup was the same as described previously (Taccola and Nistri, 2004). The spinal cord was superfused (5 ml min-1) with Krebs solution of the following composition (in mM): NaCl, 113; KCl, 4.5; MgCl2×7H2O, 1; CaCl2, 2; NaH2PO4, 1; NaHCO3, 25; glucose, 11; gassed with 95% O2-5% CO2; pH 7.4 at room temperature. All agents were bath-applied via the superfusing solution at the concentrations mentioned in the text. Recordings were obtained with glass suction electrodes (containing an Ag—AgCl pellet) filled with Krebs solution. Miniature bipolar suction electrodes were used in order to deliver single or repetitive electrical stimuli to DRs to evoke DR-DR potentials (DR-DRPs) (Kerkut and Bagust, 1995). Stimulus intensity was calculated in terms of threshold (Th), defined as the minimum intensity to elicit a detectable response in the homolateral VR.

Drugs

In the experiments in vivo, Oxytocin (Sigma) 50 μM was injected at 0.1 ml/5 g (diluted in saline) IP, 30 min before testing. Bumetanide (Sigma) solution 25 μg/ml was injected IP at the dose of 5 μmol/kg, 30 min before testing. Atosiban (Sigma) (diluted in saline) was injected at 2 μg/kg, IP, 30 min before testing. SSR126768A (gift from Sanofi-Synthelabo) diluted in saline injected at 1 mg/kg IP, 30 min before testing. Sham injections in the control group were performed with equal volumes of saline.

Statistical Analysis

Results are expressed as mean±s.e.m. Data were analyzed by a two-way analysis of variance (ANOVA) followed, when the F value was significant, by a Fischer t-test, when the time-course of the effect was compared. Significance of changes in experiments with vocalization in vivo and dorsal-dorsal responses in the isolated spinal cord in vitro was tested by the Kruskal-Wallis test (H-test). The level of statistical significance (*) was set at P<0.05.

### Results

In the present study, we used a combination of behavioral tests including thermal tail-flick assay and electrical stimulation evoked vocalizations, and electrophysiological and imaging approaches in the in vitro preparations of the spinal cord and isolated trigeminal neurons to study pain control by oxytocin and bumetanide in the newborn rats.

Analgesic Actions of Oxytocin and Bumetanide with Thermal Tail-Flick Assay

We first tested pain sensitivity in the newborn rats using a thermal tail-flick response. In this test, pain sensitivity is reciprocal to the delay in tail withdrawal from the hot water. Previous developmental studies using this test indicated that nociceptive withdrawal thresholds are low in rat pups during the first postnatal week and only increase to adult values by the second or third postnatal week (Falcon et al., 1996; Fitzgerald and Gibson, 1984; Jiang and Gebhart, 1998; Marsh et al., 1999; Teng and Abbott, 1998). We studied thermal tail-flick response in two age groups: (i) fresh newborn animals which were examined immediately, within an hour, after birth (P0) and (ii) two day-old rat pups (P2). According to previous studies, oxytocin levels are maximal during and immediately after birth, and wane during the first postnatal day, as deduced from the dynamic changes in GABA signaling in the cortical neurons (Tyzio et al., 2006). Under control conditions, newborn P0 rats withdrew their tails within 4.7±0.19 s (n=15). In P2 rats, delay in the tail withdrawal was of 2.4±0.16 s (n=15), that is nearly two times shorter than in P0 control rats (p<0.0001). Thus, pain sensitivity in the fresh newborn rats is significantly lower than in two day-old rats. We further studied whether endogenous oxytocin is involved in reduced pain in the newborn rats. To block the action of endogenous oxytocin circulating in the newborn pups we used selective blockers of oxytocin receptors atosibane (2 μg/kg, intraperiotoneal) and SSR126768A (1 μg/kg, intraperiotoneal). Both blockers caused nearly three-fold acceleration in the tail withdrawal in the P0 animals. The delays of tail withdrawal in newborn pups after oxytocin receptor blockade were similar to those seen in P2 rats under control conditions. In P2 animals, oxytocin receptor blockers did not significantly modify the tail-flick delays. These findings suggest a strong analgesic effect of endogenous oxytocin in the newborn rats, and that this effect wanes with a postnatal reduction in oxytocin levels. Systemic administration of exogendus oxytocin (1 μg/kg) resulted in a dramatic analgesic effect both in newborn and P2 rats. In the newborn rats, exogenous oxytocin could also partially reverse the effects of the competitive oxytocin receptor blockers, indicating that endogenous oxytocin levels are not saturated, and that therapeutic elevation of oxytocin levels could result in more powerful analgesia in the newborn.

Oxytocin induces a transient excitatory-to-inhibitory switch in the action of GABA on immature neurons at birth (Tyzio et al., 2006), and GABAergic mechanisms are implicated in the analgesic actions of oxytocin in adult animals (Condes-Lara et al., 2009). Lowering intracellular chloride concentration with bumetanide, selective blocker of NKCC1 co-transporter, inhibits depolarizing/excitatory actions of GABA on immature neurons similar to the effects of oxytocin. We therefore examined whether bumetanide affects pain responses in the newborn. Bumetanide (10 μM/kg) strongly delayed the tail-flick responses in both age groups, and, importantly, reversed the effect of oxytocin receptor blockers in newborn rats. Taken together, these results indicate that endogenous oxytocin and bumetanide reduces pain in newborn rats and that analgesic actions of oxytocin and bumetanide involve modulation of intracellular chloride and GABA actions in the nociceptive circuits.

Analgesic Actions of Oxytocin and Bumetanide with Thermal Vocalization Pain Assay

In the second experiment, we studied oxytocin-modulation of the pain responses by measuring vocalization evoked by electrical stimulation of the whisker pad. Animals were decerebrated at caudal midbrain levels to cut the descending oxytocin projections from the VPN to spinal cord and to prevent noxious input to the brain. Electrical stimulation of the whisker pad evoked vocalization in the neonatal rats despite of decerebration. Vocalizing response was composed of several bursts with a dominant frequency in the range from 2.7 to 5 kHz (mean frequency 3.9±0.1 kHz n=24, rat P0-2). To quantify vocalization response, we calculated scalar acusticogram integral. In agreement with the results of the thermal tail-flick assay, oxytocin receptor blocker atosiban (2 μg/kg) increased vocalization response in the fresh newborn P0 rats (to 155±28%, n=8, p=0.0003;). In P1-2 rats, injections of saline did not change the vocalization response (to 105±20%), however exogenous oxytocin reduced significantly vocal response (to 41±12%, p=0.007, n=6), and the effect was reduced by atosiban (to 84±38% from control level, p=0.025, n=6). Vocalizations were also reduced in P1-2 rats by bumetanide (to 72±16%, p=0.003, n=6;). Taken together, the results obtained in both pain models of the thermal tail-flick and electrical stimulation evoked vocalization indicate that endogenous oxytocin and bumetanide reduces pain in newborn rats and that analgesic actions of oxytocin involve modulation of intracellular chloride and GABA actions in the pain circuits.

Oxytocin Modulates GABA Signaling in the Primary Nociceptive Neurons

Because analgesic action of oxytocin in adult rats involves modulation of GABAergic control of the primary nociceptive afferents (Condes-Lara et al., 2009), we studied the effect of oxytocin on GABA responses in sensory trigeminal neurons, which detect noxious stimuli and conduct them to the spinal cord. Experiments were performed in primary cultures of trigeminal neurons dissociated from newborn rats and kept for five hours in vitro. In keeping with previous observations (Wang et al., 1994; Reichling et al., 1994), activation of GABA receptors induced robust transient increases of intracellular calcium in trigeminal neurons indicating depolarizing action of GABA and calcium entry into the cells via voltage-gated calcium channels. Application of 1 μM-oxytocin induced slow transient responses (˜60-80 s duration; not shown) and significantly reduced GABA-evoked calcium increases. The depolarizing action of GABA in sensory neurons is controlled by intracellular chloride homeostasis, in particular by the highly expressed NKCC1 membrane chloride co-transporter (Delpire and Mount, 2002). Therefore, we tested the effect of the NKCC1 blocker bumetanide on these responses. Similar to the effects of oxytocin, bumetanide suppressed the GABA receptor mediated increases in intracellular calcium.

Pain signaling in nociceptive neurons involves activation of P2X3 receptors and TRPV1 receptors (RA: REFS?). To examine whether analgesic actions of OT in vivo are mediated by modulation of P2X3 and TRPV1 receptor mediated signaling, we studied the effects of OT on the responses evoked by the agonists of these receptors in DRG neurons. Brief (2 s—long pulses at 10 min intervals) application of the selective P2X3 receptor agonist α-β-methylenATP (α-β-meATP, 10 μM; n=276 cells) and TRPV1 receptors agonist capsaicin (200 nM; n=223 cells) evoked transient, and quite stable Ca2+ increases in DRG neurons. Application of 1 μM-OT for 20-30 min did not significantly change the amplitude of these responses (n=283 and 248 cells for α-β-meATP and capsaicin, respectively). Thus, OT does not modify P2X3 and TRPV1 receptor mediated responses in DRG neurons, further supporting our hypothesis that antinociceptive effects of OT involves modulation of GABA signaling in the nociceptive neurons.

Because the results of calcium imaging suggest that oxytocin reduces depolarizing action of GABA on trigeminal neurons, we further studied the effect of oxytocin on the GABA driving force (DFGABA) using cell-attached recordings of single GABA(A) channels. DFGABA was deduced from reversal potential of the currents via GABA(A) channels (Serafini et al., 1995; Tyzio et al., 2006; Tyzio et al., 2008). In control conditions, GABA exerted strongly depolarizing action on the immature trigeminal neurons with DFGABA of 38.7±2.4 mV (n=6). In the presence of oxytocin (1 μM), DFGABA reduced to 17.7±6.7 mV (n=5; P<0.05).

Depolarizing action of GABA on the axons of primary afferents underlies primary afferents depolarization (PAD), that is a depolarizing response evoked by dorsal root stimulation in the neighboring dorsal root (Willis, 2006; Rudomin and Schmidt, 1999). Therefore, in the next experiments we have studied the effect of oxytocin on PAD in the in vitro isolated spinal cord preparations obtained from newborn rats. In control conditions, the electrical stimulation of DRL4 evoked PAD in the homolateral DRL5 of 0.74±0.50 mV (n=13), which was completely suppressed by the GABAA receptor antagonist bicuculline (10 μM, data not shown). Oxytocin (1 μM) alone reduced the peak of DR-DRPs to 93.6±5.6% of control, an effect that was then reverted to 106.0±20.8% by adding atosiban (10 μM), while atosiban alone increased the peak to 112.7±9.1% (p=0.016, n=8). In five of these preparations, the addition of bumetamide (20 μM) to atosiban (10 μM) reduced the peak to 66.6±13.8% with respect to control values (p=0.006, n=5). On the contrary, in five preparations in which recordings were taken between 12 and 24 hours after birth, 10 μM of atosiban were not able to significantly decrease peak of DR-DRPs (90.0±9.8% of control) while the reduction induced by oxytocin was still observed (77.8±21.6% of control). Thus, the results of calcium imaging and cell-attached measurements of GABA responses, and the results of the pharmacological analysis of PAD indicate that oxytocin and bumetanide reduces depolarizing action of GABA in sensory trigeminal and dorsal root ganglion neurons.

### REFERENCES

Throughout this application, various references describe the state of the art to which this invention pertains. The disclosures of these references are hereby incorporated by reference into the present disclosure.

Derek L-J (1999) Fundamentals of Obstretics and Gynaecology.

Khazipov R, Tyzio R, Ben Ari Y (2008) Effects of oxytocin on GABA signalling in the foetal brain during delivery. Prog Brain Res 170:243-57.:243-257.

Tyzio R, Cossart R, Khalilov I, Minlebaev M, Hubner C A, Represa A, Ben Ari Y, Khazipov R (2006) Maternal Oxytocin Triggers a Transient Inhibitory Switch in GABA Signaling in the Fetal Brain During Delivery. Science 314:1788-1792.

1. A compound which inhibits the importation of chloride into neurons or a compound which improve the outflow of chloride from neurons formulated for use in a method for alleviating pain and stress in fetus and newborn.

2. A compound according to the claim 1, wherein said compound inhibits the NKCC co-transporter or activates the KCC co-transporter.

3. A compound according to the claim 1 wherein said compound is an antagonist of NKCC1.

4. A compound according to the claim 1 wherein the compound is a diuretic.

5. A compound according to the claim 1 wherein the compound is bumetanide.

6. A pharmaceutical composition for use in for use in a method for alleviating pain and stress in a fetus or newborn comprising a compound according to claim 1 and a pharmaceutically acceptable carrier, said composition formulated for delivery to said fetus or newborn.

7. A method for screening a drug for use in a method for alleviating pain and stress in a fetus or newborn comprising the steps of:
a. providing neurons expressing NKCC or KCC on their surface;
b. incubating said cells with a candidate compound;
c. determining whether said candidate compound binds to and inhibits NKCC or binds to and activates KCC; and
d. selecting the candidate compound that binds to and inhibits NKCC or binds to and activates KCC.

8. A method for alleviating pain and stress in a fetus or newborn, comprising:
administering to a fetus or newborn subject in need thereof a compound which inhibits the importation of chloride into neurons or a compound which improves the outflow of chloride from neurons.

9. The method of claim 8 wherein said compound inhibits the NKCC co-transporter or activates the KCC co-transporter.

10. The method according to claim 8 wherein said compound is an antagonist of NKCC1.

11. The method according to claim 8 wherein the compound is a diuretic.

12. The method according to claim 8 wherein the compound is bumetanide.
